(19) United States
(12) Patent Application Publication
(10) Pub. No.: US 2007/0014835 A1
(43) Pub. Date: Jan. 18, 2007

(54) Blood purifier

(75) Inventor: Masahiko Takahashi, Tokyo (JP)

Correspondence Address: GREENBLUM & BERNSTEIN, P.L.C., 1950 ROLAND CLARKE PLACE, RESTON, VA, 20191, US

(73) Assignee: Amity Corporation, Tokyo (JP)

(21) Appl. No.: 11/213,733

(22) Filed: Aug. 30, 2005

(30) Foreign Application Priority Data
Jun. 15, 2005 (JP) 2005-174634

(51) Int. Cl. A61K 33/24; A61K 9/70

(52) U.S. Cl. 424/443; 424/617

(57) ABSTRACT

A blood purifier includes a first material selected from a group made of copper material, platinum material and molybdenum material, a second material selected from a group made of black silica material, radon material, germanium material, neodymium material and silica material and a titanium oxide material. The blood purifier at least includes the titanium oxide material for a contact part with a user's outer skin. The contact part is made of the second material and a mix-coated layer that contains the titanium oxide material. The contact part is also made of a surface coating layer that contains the titanium oxide material, having a middle coating layer that contains the second material and a cover base placed in this order underneath the surface coating layer.

### BACKGROUND OF THE INVENTION

1. Field of the Invention

The present invention relates to a blood purifier that purifies blood by making contact with a human body, for example, through the outer skin.

2. Description of Related Art

Today, the number of modern diseases is increasing due to westernized dietary habits. It has been discovered that blood conditions have a great effect on heath. Given this situation, a plate-like body that makes blood mildly alkaline by generating negative ions has been proposed (Refer to Related Art 1).

The plate-like body is coated with an embrocation containing finely powdered tourmaline ore. Therefore, the plate-like body is able to effectively generate negative ions, thereby making blood mildly alkaline with the negative ions.

Although the plate-like body changes acidic blood that can cause various diseases to mildly alkaline blood, it is not capable of purifying blood itself by normalizing blood conditions, whereby blood component test results approach the normal average values.

Related Art 1] Japanese Laid-Open Publication 2002-264277

### SUMMARY OF THE INVENTION

The purpose of the present invention is to provide a blood purifier that purifies blood by making contact with a human body through the outer skin.

The present invention includes the first material that is selected from a group made of copper material, platinum material and molybdenum material, the second material that is selected from a group made of black silica material, radon material, germanium material, neodymium material and silica material, and a titanium oxide material. The present invention uses at least the titanium oxide material for a contact part that comes in contact with the user's skin.

The first material includes a mono metal or an alloy selected from the group made of copper material, platinum material and molybdenum material in solid or powder form. In addition, the alloy may be a binary alloy, ternary alloy or other multiple alloys. The contact may be a made directly or indirectly with the user's outer skin through a fabric-like substance. The contact part may be surface, line or point that comes in contact with the user's skin directly or indirectly.

The present invention is thus capable of purifying blood as the user's skin comes in contact with the contact surface. In addition, the blood purification effects include cases where blood conditions are normalized as a result of changes in the test values of some particular components identified in a blood component test to approach their normal values. Also included are cases where intertwined red blood cells identified in a blood micrograph (hereafter referred to as “rouleaux formation”) are separated and released from each other, or cases where blood conditions are normalized as a result of changes in the shapes of red blood cells to near-globular shapes.

In the embodiment of the present invention, the contact part may be made of a layer containing the second material and the titanium oxide material. The layer containing the second material and the titanium oxide material is made of a material that mixes the second material and the titanium oxide material, the second material being selected from a group of black silica material, radon material, germanium material, neodymium material and silica material. The layer may be formed with solvent, paint or adhesive. The contact part may be a part or all of the surface of the first material. Accordingly, it is possible to easily configure the blood purifier, thereby purifying users' blood.

In the embodiment of the present invention, the contact part may be made of a titanium oxide layer that contains the titanium oxide material, having the second layer containing the second material and the first material placed in this order underneath the titanium oxide layer. Accordingly, desired blood purification effects may be obtained by specifying the thickness of each layer.

In addition, in the embodiment of the present invention, the first material in powder form, the second material, the titanium oxide material and a plastic material may be mixed to form a composite material. The plastic material may be, for example, a thermosetting material or a thermoplastic material. Specifically, the plastic material may be a resin, a glass, a ceramic, etc. In the above-noted embodiment, the blood purifier may be made by mixing a liquid or gelatinous plastic material with the first material in powder form, the second material and the titanium oxide material to form a composite material, which then solidifies.

Accordingly, the first material in powder form, the second material and the titanium oxide material are all mixed as the composite material. Therefore, even when the surface is worn out, the composition ratio remains the same, thereby improving the product durability of the blood purifier. This also makes it easier to form a complex-shaped blood purifier

In the embodiment of the present invention, the plastic material may be made of a resin material. The resin material may be a synthetic resin or a natural resin, thereby making it possible to form a complex-shaped blood purifier more easily. In addition, a resin composite material is suited for mass production, thus making it possible to lower the production costs for the blood purifier.

In the embodiment of the present embodiment, the copper material may be made of an alloy that contains copper with a mass ratio of 40% or more. Since many kinds of alloys may be used, it is possible to obtain such blood purification effects and make material selections suited for specific purposes, thereby improving users' satisfaction level.

In the embodiment of the present invention, the blood purifier may be provided on the body surface of an electronic device. Such electronic devices include cell phones, personal computers, personal computer input devices such as mice, portable compact music devices such as MD players, compact camera devices such as digital video cameras, voice input devices such as microphones, portable game devices and input devices for portable game devices. Such bodies include bodies themselves, operating units provided on a part of a body, etc. Accordingly, blood purification effects may be obtained by using such electronic devices.

In the embodiment of the present invention, at least a part of the body of an electronic device is made of the copper material or a platinum material, providing the contact part of the blood purifier on the body surface of the electronic device. Accordingly, blood purification effects may be obtained by using such an electronic device.

In the embodiment of the present invention, the blood purifier may be formed on the surface of a daily item. Such daily items include accessories, such as rings, necklaces and a pair of glasses, kitchen items, such as knives, forks, a pair of chopsticks and plates, and personal articles such as pens, lighters and cases. Accordingly, blood purification effects may be obtained by using such daily items.

In addition, in the embodiment of the present invention, the fibrous-formed blood purifier may also be woven into a fabric item. Such fabric items include clothes such as underwear, shirts and pants, and accessories such as supporters, mufflers and hats. Accordingly, blood purification effects may be obtained by wearing such clothes or accessories.

According to the present invention, it is possible to purify the user's blood by making contact with a contact part.

### BRIEF DESCRIPTION OF THE DRAWINGS

The above, and other objects, features and advantages of the present invention will be made apparent from the following description of the preferred embodiments, given as nonlimiting examples, with reference to the accompanying drawings in which:

FIGS. 1(a) and 1(b) illustrate a blood purification effect cell phone having a blood purification sticker attached;

FIG. 2 illustrates the blood purification effect cell phone having the blood purification sticker attached;

FIG. 2(a) is an enlarged detail of a portion of FIG. 2;

FIGS. 3(a) and 3(b) show a breakdown of blood component test results for test subjects;

FIGS. 4(a)-4(d) show blood micrographs of test subject A;

FIG. 5(a)-5(d) show blood micrographs of test subject B;

FIG. 6 shows an enlarged central vertical cross-sectional view of the blood purification effect cell phone according to the second embodiment;

FIG. 6(a) is an enlarged detail of a portion of FIG. 6;

FIG. 7 shows an enlarged central vertical cross-sectional view of the blood purification effect cell phone according to the third embodiment.

### DETAILED DESCRIPTION OF THE INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description is collected with the drawings making apparent to those skilled in the art how the forms of the present invention may be embodied in practice.

The fist embodiment of the present invention is described in the following with reference to the drawings.

### First Embodiment

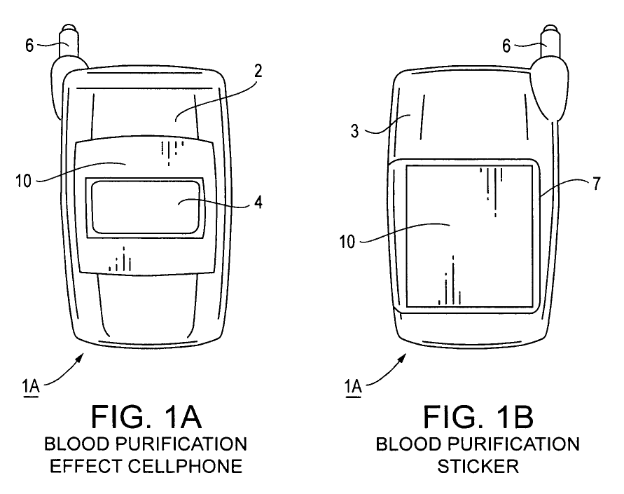

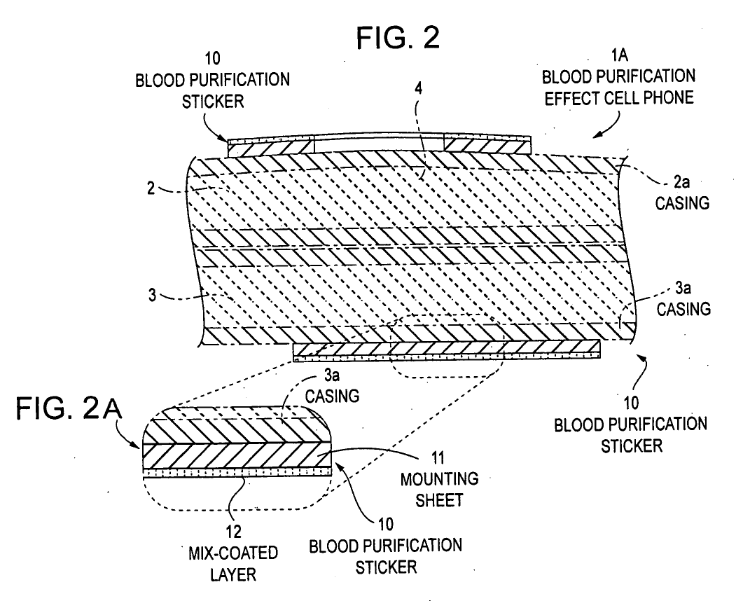

Blood purification sticker 10 and blood purification effect cell phone 1A are illustrated with reference to FIGS. 1 and 2. FIG. 1 illustrates blood purification effect cell phone 1A having blood purification sticker 10 attached. FIG. 2 illustrates an enlarged central vertical cross-sectional view of blood purification effect cell phone 1A. In addition, FIG. 1(a) shows a top plain view of cell phone 1 folded; FIG. 1(b) shows a bottom view of cell phone 1 folded.

As shown in FIG. 1, cell phone 1 is equipped with receiver 2 and transmitter 3 in an upper part attached by hinge 5 that enables an easy opening and closing. Cell phone 1 is equipped with a display and a receiving speaker, which are not shown in the drawings, on the inner side of receiver 2 and with a button and a transmitting microphone, which are not shown in the drawings, on the inner side of transmitter 3.

As shown in FIG. 1(a), receiver 2 folded is equipped with a top-view rectangle LCD (Liquid Crystal Display) 4. As shown in FIG. 1(b), transmitter 3 includes battery 7 and antenna 6. Antenna 6 is configured to be extendable.

As shown in FIG. 1(a), on casing 2a (FIG. 2) of receiver 2, blood purification sticker 10 is attached to LCD 4 with an opening in roughly its center to fit the size of LCD 4. Furthermore, as shown in FIG. 1(b), blood purification sticker 10 is attached to casing 3a (FIG. 2) of battery 7 of transmitter 3.

The top plain view size of blood purification sticker 10 is approximately 35 mm×45 mm. As shown in the enlarged detailed FIG. 2a, blood purification sticker 10 includes sticker board 11 and mix-coated layer 12. Sticker board 11 is a copper (the first material) thin film, with one side adhering to cell phone 1. In addition, the opposite side to the adhering side of sticker board 11 is polymerized with mix-coated layer 12.

Mix-coated layer 12 is a mix of water-based urethane coatings, powdery black silica (the second material) and titanium oxide material, and is applied to the surface of sticker board 11. In this example, a brush paint application is adopted as the application method. However, the application method is not limited to this, and such methods as spray, baking, and printing methods may be also applied.

The thickness of mix-coated layer 12 is smaller than or almost the same as that of sticker board 11. As for the composition ratio of the black silica and titanium oxide, the mass ratio of the black silica to the total mass of mix-coated layer 12 may range from 2 to 12%, while the mass ratio of the titanium oxide to the total mass may range from 2 to 25%.

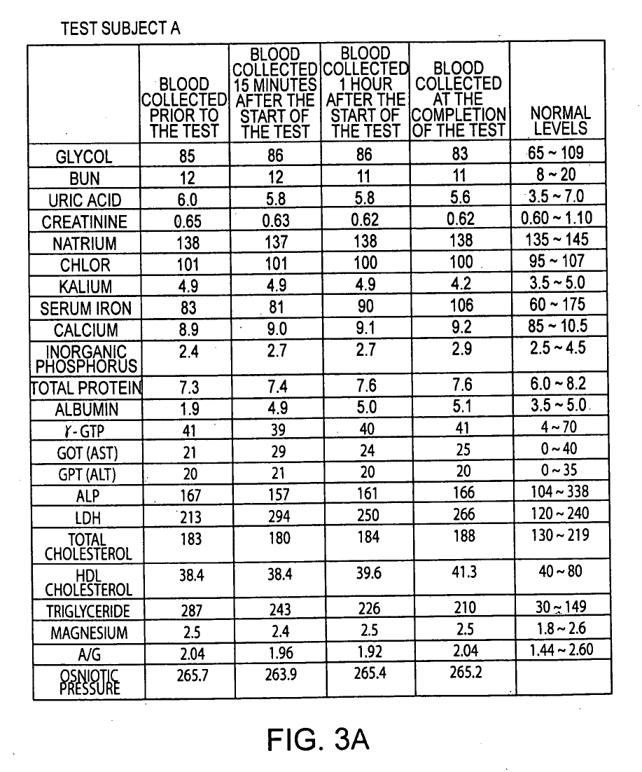

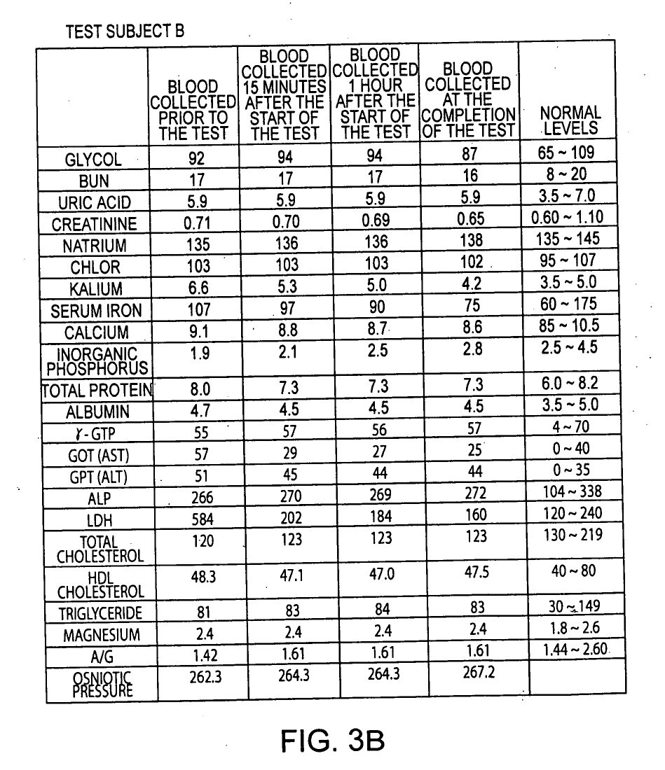

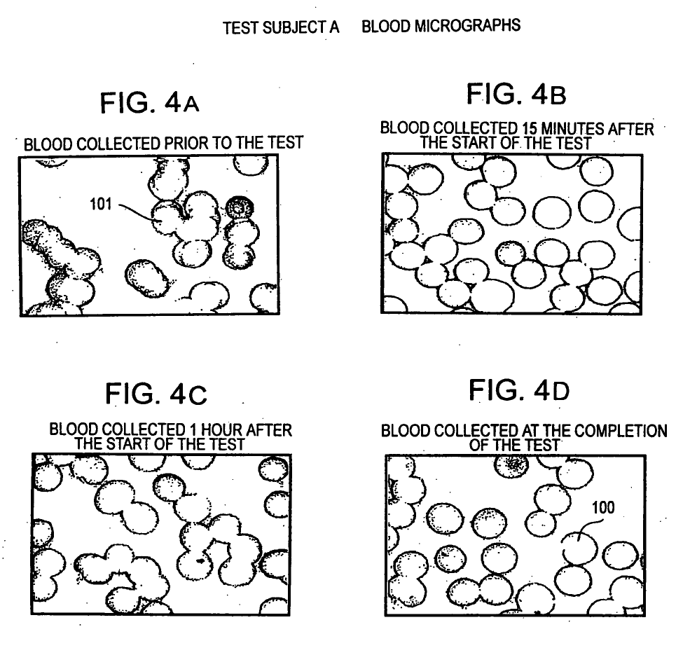

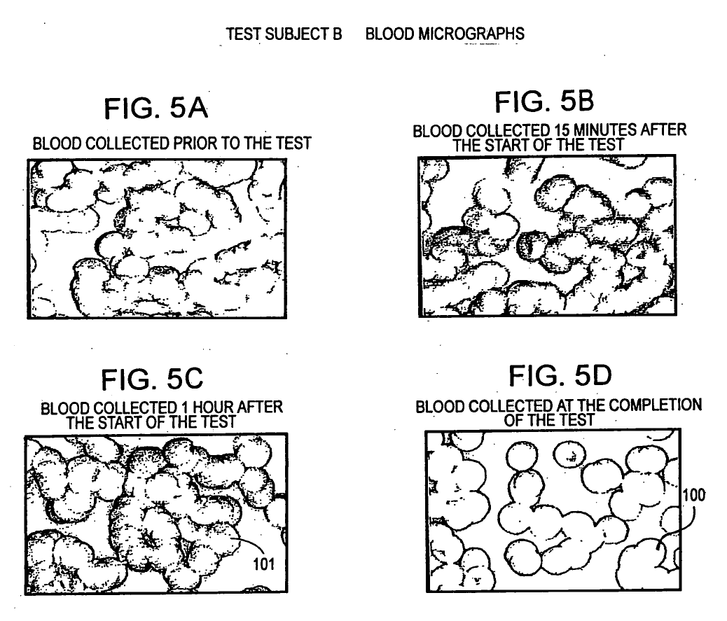

Next, a blood purification effect verification test using blood purification effect cell phone 1A and the test results are described with reference to FIGS. 3, 4 and 5. FIG. 3 shows a breakdown of blood component test results for test subjects. FIGS. 4 and 5 illustrate blood micrographs of the test subjects. The blood purification effect verification test starts with a survey asking a plurality of test subjects regarding their life styles, including their smoking histories, drinking habits and daily sleeping conditions. The test subjects are then asked to repeatedly use and then store blood purification effect cell phone 1A at predetermined time intervals. At each time interval, the blood of each test subject is collected for a blood composition test and blood micrograph, after which the results are compared.

When in use, receiver 3 of blood purification effect cell phone 1A is opened and held with one hand touching its outer side. When stored, blood purification effect cell phone 1A is folded and stored in a pocket of pants or a shirt. Therefore, when in use, blood purification sticker 10 attached to transmitter 3 is touching the hand, and when stored, one of blood purification sticker 10 attached to transmitter 3 and receiver 2 is indirectly touching the body through clothing.

Blood purification effect cell phone 1A is used for 15 minutes after the start of the test (hereinafter referred to as “first use”), and then stored for 30 minutes (hereinafter referred to as “first storage”). Blood purification effect cell phone 1A is then used for the second use of 15 minutes (hereinafter referred to as “second use”), stored for the second storage of 1 hour and 45 minutes (hereinafter referred to as “second storage”) and used for the final use of 15 minutes (hereinafter referred to as “final use”).

Furthermore, a blood test uses four collected blood samples, each time collecting about 30 cc blood: prior to the test (hereinafter referred to as “blood collected prior to the test”; after the first use ends, which is 15 minutes after the start of the test (hereinafter referred to as “blood collected 15 minutes after the start of the test”; after the second use ends, which is one hour after the start of the test (hereinafter referred to as “blood collected one hour after the start of the test”); and after the final use ends, which is three hours after the start of the test (hereinafter referred to as “blood collected at the completion of the test”). In this blood purification effect verification test, the blood purification effects for two test subjects are compared.

The following describes the results of the blood purification effect verification test. FIG. 3 shows the results of the blood component test that was conducted 4 times for each test subject and normal values for each component. Each table of blood components shows component items in the left side column and times of blood collection for the blood test in the top row. FIG. 3(a) shows the test values of the blood component test for test subject A. Test subject A is an over-weight male adult smoker. The results of the blood purification effect verification test for test subject A confirmed that, in a plurality of component items, the test values approached the median values within the normal control ranges.

Particularly, as shown in FIG. 3(a), of the test values for individual blood components items in the blood collected prior to the test, inorganic phosphorus and HDL cholesterol shown in the heavy-line rows were slightly lower than their normal value ranges and neutral lipid was greatly higher than its normal value range.

Inorganic phosphorus fell within the normal value range as a result of an increase in the test values for the blood collected 15 minutes after the start of the test. It was confirmed that the test values for the blood collected at the completion of the test showed a further increase, thus approaching the median value within the normal value range. It was also confirmed that the test value for HDL cholesterol showed an increase in the blood collected after 15 minutes after the start of the test and further increased in the blood collected at the completion of the test to come within the normal value range.

Further, the test value for neutral lipid started to drop 15 minutes after the start of the test. Although the test value for the blood collected at the completion of the test did not drop to come within the normal value range, the test value stood substantially closer to the normal value. Further, besides the above items, the test confirmed that uric acid, kalium and serum iron were within the normal value ranges before the start of the test and even came closer to the median values within the normal value ranges.

The above-noted test results confirmed homeostasis, whereby the values of a plurality of blood components, including those whose values were outside their normal value ranges, approached the median values within their normal value ranges. Homeostasis means a process/mechanism in which the body reverts to its most desirable conditions.

The following describes the results of the blood component test for test subject B as shown in FIG. 3(b). Test subject B is a slightly over-weight adult male smoker. The results of the blood purification effect verification test for test subject B confirmed that, in a plurality of component items, the test values approached the median values within their normal control values.

Particularly, as shown in FIG. 3(b), it was confirmed that prior to the test, six component items in the heavy-line rows, i.e. kalium, inorganic phosphorus, GOT (AST), GPT (ALT), LDH and total cholesterol, showed the test values outside their normal value ranges. More specifically, the test values for inorganic phosphorus and total cholesterol were below their normal value ranges, and the test values for the other items, i.e. kalium, GOT (AST), GPT (ALT), and LDH, were above their normal value ranges.

The test confirmed that the test value of kalium started to decline in the blood collected 15 minutes after the start of the test, continued to decline to reach the high end of its normal value range in the blood collected one hour after the start of the test, and further declined to approach the median value within its normal value range in the blood collected at the completion of the test.

Further, the test confirmed that the test value for inorganic phosphorus started to increase in the blood collected 15 minutes after the start of the test, continued to increase to reach the lower end of its normal value range in the blood collected one hour after the start of the test, and further increased to approach the median value of its normal value range in the blood collected at the completion of the test.

Further, the test confirmed that the test value for GOT (AST) greatly dropped to come within its normal value range in the blood collected 15 minutes after the start of the test, and further dropped to approach the median value of its normal value range in the blood collected at the completion of test. The test further confirmed that the test value for GPT (ALT) showed a decrease in the blood collected 15 minutes after the start of the test and further decreased to approach its normal value range in the blood collected at the completion of the test, although the test value did not come within its normal value range itself.

Further, the test confirmed that the test value for LDH started to drop to come within its normal value range in the blood collected 15 minutes after the start of the test and further declined in the blood collected at the completion of the test. Moreover, it was confirmed that the test value for total cholesterol started to increase in the blood collected 15 minutes after the start of the test, but did not come within its normal value range in the end. Besides the above-noted items, the test values for natrium, total protein, albumin and ALP were within their normal value ranges in the blood collected prior to the test, and approached the median values within the normal value ranges in the blood collected 15 minutes after the start of the test and thereafter.

The test results for test subject B confirmed homeostasis for a plurality of blood components besides blood components whose test values were outside their normal value ranges, whereby the test values approached the median values within the normal value ranges. Although it is not described in the FIG. 3 data, the test also confirmed that the blood pressure level of a hypertensive test subject declined to approach the normal value range after the completion of the test.

The following describes the blood micrographs of test subject A as shown in FIG. 4. FIG. 4 shows blood micrographs of: (a ) blood collected prior to the test (top left), (b) blood collected 15 minutes after the start of the test (top right), (c) blood collected one hour after the start of the test (bottom left), and (d) blood collected at the completion of the test. Each blood micrograph shows erythrocyte 100 blood cells in the blood.

Normal erythrocyte 100 blood cells are separated and independent from each other in near-globular shapes. In contrast, as shown in FIG. 5(a), the test confirmed that most of erythrocyte 100 blood cells in test subject A prior to the test were intertwined with each other, instead of being independent, by rouleau formation to form erythrocyte aggregate 101. Moreover, the degree of intertwinement for erythrocyte 100 blood cells was so great that the shape of each individual erythrocyte 100 blood cell could not be confirmed. In general, an increase in the amount of erythrocyte aggregate 101 by rouleau formation in blood is believed to lead to an increase in blood viscosity. Therefore, the blood of test subject A was not in their desirable conditions in terms of health.

As shown in FIG. 4(b) illustrating the blood micrograph taken 15 minutes after the start of the test, although erythrocyte aggregate 101 still existed, the degree of intertwinement for erythrocyte 100 blood cells was lessened and the shape of each individual erythrocyte 100 blood cell was discernible. As shown in FIG. 4(c) illustrating the blood micrograph taken one hour after the start of the test, it was confirmed that the degree of intertwinement for erythrocyte 100 blood cells was further lessened. As shown in FIG. 4(d) illustrating the blood micrograph taken three hours after the start of the test, although erythrocyte aggregate 101 still existed, it was confirmed that the number of such aggregates decreased, with many unconnected individual erythrocyte 100 blood cells detected. Moreover, many erythrocyte 100 blood cells were found in near-globular shape.

To summarize the change over time as shown in the above-noted blood micrographs, it was confirmed that the degree of rouleau formation decreased as the test progressed with erythrocyte 100 blood cells gradually separated and released from each other to approach their normal state in near-globular shape. A decrease in blood viscosity was thus confirmed, although it is not shown in the test results of the blood component test in FIG. 3.

The following describes the blood micrographs for test subject B as shown in FIG. 5. Same as FIG. 4, FIG. 5 shows blood micrographs of: (a) blood collected prior to the test, (b) blood collected 15 minutes after the start of the test, (c) blood collected one hour after the start of the test, and (d) blood collected at the completion of the test.

As shown in FIG. 5(a) illustrating the blood micrograph prior to the test, the test confirmed that erythrocyte 100 blood cells for test subject B prior to the test were intertwined with each other instead of being independent from each other, forming one instance of erythrocyte aggregate 101. The number of blood erythrocyte 100 blood cells forming the instance of erythrocyte aggregate 101 and the degree of intertwinement for each erythrocyte 100 blood cell were greater than those of test subject A, indicating a very high degree of blood viscosity.

As shown in FIG. 5(b) illustrating the blood micrograph taken 15 minutes after the start of the test, it was confirmed that the degree of intertwinement for erythrocyte 100 blood cells in erythrocyte aggregate 101 was lessened. As shown in FIG. 5(c) illustrating the blood micrograph taken one hour after the start of the test, it was confirmed that the degree of intertwinement for erythrocyte 100 blood cells was lessened to such a degree that the shape of each individual erythrocyte 100 blood cell was discernible. As shown in FIG. 5(d) illustrating the blood micrograph taken three hours after the start of the test, although erythrocyte aggregate 101 was not dissolved, it was confirmed that the degree of intertwinement for erythrocyte 100 blood cells was further lessened, with single independent erythrocyte 100 blood cells detected, though small in number.

To summarize the change over time as shown in the above-noted blood micrographs, it was confirmed that the rouleau formation was gradually dissolved as erythrocyte 100 blood cells were separated from each other to approach their normal state, although the pace of the change was slower compared with that for test subject A shown in FIG. 4.

The test results of the blood purification effect verification test using blood purification effect cell phone 1A confirmed that homeostasis was achieved, through blood purification sticker 10 attached to receiver 2 and transmitter 3 that comes in contact with the outer skin, in a plurality of blood components besides those blood components whose test values were outside their normal value ranges. Although the pace of such changes varied across the test subjects, the rouleau formation was dissolved as blood cells were separated from each other to approach their normal state. This proves the blood purification effects on user blood that are caused when blood purification sticker 10 comes in contact with the outer skin.

As described above, blood purification effects can be easily obtained by using blood purification effect cell phone 1A (FIG. 1), which is created by attaching blood purification sticker 10 (FIG. 1) to cell phone 1 (FIG. 1). In addition, blood purification sticker 10 may serve an ornamental purpose for blood purification effect cell phone 1A while gaining blood purification effects, by modifying the sticker to a desired shape when attaching it to cell phone 1.

### Second Embodiment

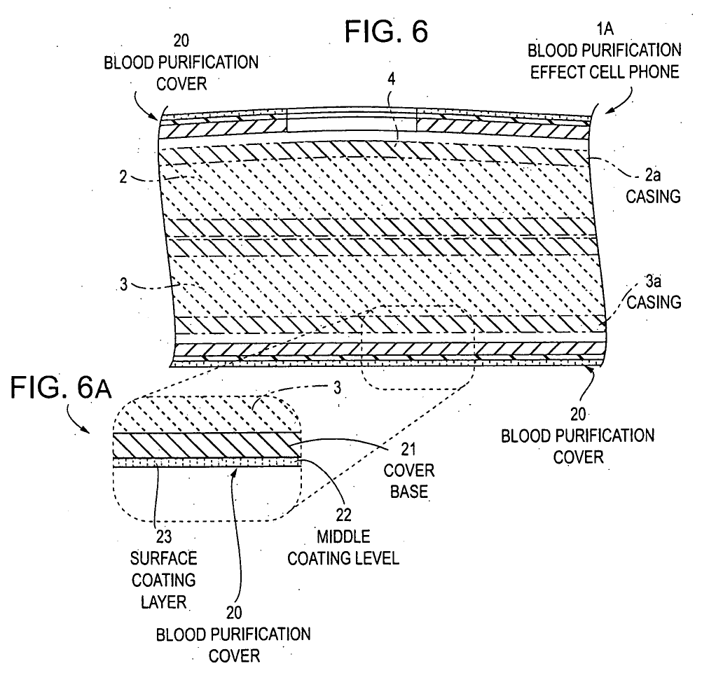

The following describes blood purification effect cell phone 1A in the second embodiment with reference to FIG. 6 showing an enlarged central vertical cross-sectional view of blood purification effect cell phone 1A. Blood purification effect cell phone 1A described in the second embodiment as shown in FIG. 6 includes blood purification cover 20 attached to the outer surfaces of casing 2a of receiver 2 and casing 3a of transmitter 3 of ordinary cell phone 1 as shown in FIG. 1.

Blood purification cover 20 is shaped to fit the outer shapes of casing 2a and casing 3a and covers the entire surface of each casing. In addition, blood purification cover 20 may be configured to be detachable. In FIG. 6, there is a gap between the outer sides of casing 2a and casing 3a and the inner sides of blood purification cover 20 for the purpose of clearly illustrating the attachment condition of blood purification cover 20. These parts may be tightly attached with no gap.

As shown in the enlarged detailed FIG. 6(a), blood purification cover 20 includes middle coating layer 22 that contains black silica on the outer surface of copper cover base 21 with sufficient strength, and surface coating layer 23 that contains titanium oxide on the outer surface of middle coating layer 22 in this order, forming a three-layer structure. Furthermore, middle coating layer 22 and surface coating layer 23 may be formed by such application methods as brush paint, spray paint, baking, and printing.

Accordingly, blood purification effect cell phone 1A may be easily created by attaching blood purification cover 20 to existing cell phone 1. Compared with blood purification sticker 10 (FIG. 2), blood purification cover 20 is made of copper cover base 21 with sufficient strength, thereby improving the product durability of blood purification effect cell phone 1A. It is also possible to gain desired blood purification effects with ease by adjusting the thickness of middle coating layer 22 and surface coating layer 23. In this case, the same blood purification effects as those in the first embodiment may be obtained.

### Third Embodiment

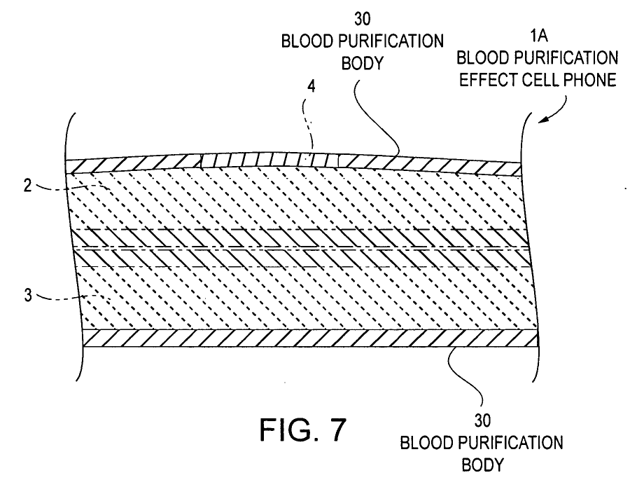

The following describes blood purification effect cell phone 1 B in the third embodiment with reference to FIG. 7 showing the enlarged central vertical cross-sectional view of blood purification cell phone 1B. Blood purification effect cell phone 1B is equipped with casing 2a of receiver 2 and casing 3a of transmitter 3 that are themselves made of a powdery copper material and a composite resin material containing black silica and titanium oxide, thereby constituting blood purification body 30.

The resin does not need to be a particular kind and any proper synthetic resin serves the purpose. In addition, it is possible to use a thermoplastic material or a thermosetting material, such as ceramic and glass, for example, as substitute for resin.

Accordingly, users can gain blood purification effects by using blood purification effect cell phone 1B without attaching blood purification sticker 10 and the like. Furthermore, it is difficult to produce blood purification effect cell phone 1B in a shape that would be impossible to attain by attaching or modifying blood purification sticker 10 (shown in FIG. 2) or blood purification cover 20 (shown in FIG. 6).

Moreover, the use of blood purification body 30, which is made of a mix of the powdery copper material, the black silica material and the titanium oxide material, improves the product durability since the combination ratio of the materials remains the same, even when wear occurs on the surface.

In other embodiments, it is possible to use in a variety of daily items a composite resin material containing powdery copper, black silica, and titanium oxide, or mix-coated layer 12 (FIG. 2) on the outer surface of a copper material, or middle coating layer 22 and surface coating layer 23 provided in this order on the outer surface. Such daily items include: daily-use tableware such as spoons, forks, and plates; personal articles such as lighters and handles of bags; accessories such as wrist watches and necklaces; miscellaneous goods such as portable cases; and household items such as chairs and beds. Blood purification sticker 10 may also be attached to electronic devices, including portable compact music devices, personal computers, and input devices such as computer mice.

The above-noted embodiments represents illustrative examples. Without being limited to these embodiments, users can use the present invention for the entire body or a part of an object that comes in contact directly or indirectly with their outer skin. Accordingly, users can gain blood purification effects every time they use the item in their daily life.

Furthermore, clothes such as underwear, shirts, and pants, or other accessories such as supporters, mufflers, and hats may be configured with blood purification effect fabrics alone or with normal fabrics woven with blood purification effect fabrics, which are made either of mix-coated layer 12 (FIG. 2) adhered to the outer surface of a fibrous copper material, or of middle coating layer 22 and surface coating layer 23 adhered to the outer surface of the fibrous copper material in this order.

Accordingly, users are able to obtain blood purification effects when wearing such clothes or accessories as the blood purification effect fabric woven into the contact surface comes into contact with their outer skin. Therefore, constant blood purification effects may be obtained while such items are in use.

In addition, in any of the above-noted embodiments, a platinum material, a molybdenum material or a composite material made of platinum and copper or molybdenum and copper may be used as substitute for the copper material. Similarly, in any of the above-noted embodiments, radon, germanium, neodymium, or silica may also be used as substitute for the black silica.

In the embodiments of the present invention, the blood purifier is embodied as blood purification sticker 10, blood purification cover 20, casing 2a or casing 3a of blood purification effect cell phone 1B. In the same manner, the contact part is embodied as mix-coated layer 12, surface coating layer 23 and blood purification body 30. The layer that contains the second material and the titanium oxide material is embodied as mix-coated layer 12; the second material layer is embodied as middle coating layer 22; the titanium oxide layer is embodied as surface coating layer 23; the body is embodied as blood purification body 30; electronic devices are embodied as cell phone 1, compact portable music devices, personal computers and input devices such mice; the complex material is embodied as a resin-made complex material containing a powdery copper material, black silica, and titanium oxide; daily items are embodied as daily-use tableware such as spoons, forks and plates, personal articles such as lighters and handles of bags, accessories such as wrist watches and necklaces, miscellaneous items such as portable cases, and household items such as chairs and beds; the fabric is embodied as clothes such as underwear, shirts and pants and accessories such as supporters, mufflers, and hats. The present invention is not limited to the above-noted embodiments and may be realized in many other embodiments.

The present disclosure relates to subject matter contained in priority Japanese Application No. 2005-174634, filed on Jun. 15, 2005, which is herein expressly incorporated by reference in its entirety.

Although the invention has been described with reference to an exemplary embodiment, it is understood that the words that have been used are words of description and illustration, rather than words of limitation. Changes may be made within the purview of the appended claims, as presently stated and as amended, without departing from the scope and spirit of the invention in its aspects. Although the invention has been described with reference to particular means, materials and embodiments, the invention is not intended to be limited to the particulars disclosed. Rather, the invention extends to all functionally equivalent structures, methods, and uses such as are within the scope of the appended claims.

What is claimed is:

1. A blood purifier comprising:
a first material selected from a group made of copper material, platinum material and molybdenum material;
a second material selected from a group made of black silica material, radon material, germanium material, neodymium material and silica material, and
a titanium oxide material, wherein a contact part that comes in contact with a user's skin at least includes said titanium oxide material.

2. The blood purifier according to claim 1, wherein the contact part is made of a layer containing said second material and said titanium oxide material.

3. The blood purifier according to claim 1, wherein the contact part is made of a titanium oxide layer containing said titanium oxide material, and wherein said second material and said first material are placed in this order underneath said titanium oxide layer.

4. The blood purifier according to claim 1, wherein the contact part is made of a composite material mixing said first material in powder form, said second material, said titanium oxide material and a plastic material.

5. The blood purifier according to claim 4, wherein the plastic material is made of a resin material.

6. The blood purifier according to claim 1, wherein the copper material is made of an alloy containing copper with a mass ratio of 40% or more.

7. An electronic device comprising the blood purifier according to claim 1 on the surface of the body.

8. The electronic device, wherein at least one part of the body is made of said first material and wherein the contact party of the blood purifier described in claim 2 is provided on the body surface.

9. A daily item comprising the blood purifier described in claim 1 on its surface.

10. A fabric element comprising the woven blood purifier described in claim 1 in fibrous form.
